(19) United States
(12) Patent Application Publication
(10) Pub. No.: US 2020/0037867 A1
(43) Pub. Date: Feb. 6, 2020

(54) APPARATUS AND METHOD FOR AIR-ASSISTED MIRROR CLEANING

(71) Applicants: Walter Pace Plant, Temple, TX (US); Brian Corey Slighly, Round Rock, TX (US)

(72) Inventors: Walter Pace Plant, Temple, TX (US); Brian Corey Slighly, Round Rock, TX (US)

(21) Appl. No.: 16/052,740

(22) Filed: Aug. 2, 2018

(51) Int. Cl. A61B 1/253; A61B 1/12; A61B 1/00

(52) U.S. Cl. CPC A61B 1/253; A61B 1/00091; A61B 1/126

(57) ABSTRACT

A dental instrument with a substantially hollow body is attached to a flexible tube and the flexible tube is attached to the supply outlet of a standard air-water syringe at the dentist's workstation. A locking mechanism may be placed in a first position, with the water supply button of the air-water syringe being essentially disengaged and with the air supply button being unaffected. The locking mechanism may then be rotated from the first position to a second position and the raised portion of the locking mechanism being positioned over the air supply button, thereby “locking” the air supply button in the “on” position. With the air supply button locked in the “on” position, the air supply from the air-water syringe is delivered through the flexible tubing, through the hollow body of the dental instrument, and then directed across the face of the mirrored surface of the dental instrument.

### BACKGROUND OF THE INVENTION

### 1. Field of the Invention

The present invention relates generally to the field of dentistry and more specifically relates to a apparatus and method for improvement of dental tools.

### 2. Related Art

One of the most important tools used by dental professionals is the simple handheld mirror. This mirror is used in virtually every procedure so as to provide the dental professional with a complete view of the inside of the patient's mouth. Handheld mirrors generally comprise a small circular piece of reflective glass held in a frame and affixed to an elongated handle, where it is held by the dental professional performing the procedure.

A well-known problem associated with the standard dental instrument is that the mirror frequently becomes covered with water, dust, or other material to the point that the dentist's view of the work surface is obstructed. The dentist must then withdraw the mirror from the patient's mount, clean the mirror, place the mirror back in the patient's mouth, get re-oriented and resume the procedure. In procedures in which a rotary drilling, sanding, or polishing device is used, the problem of obstructing material accumulating on the mirror is particularly acute because, in addition to the fragments that break away from the patient's teeth and gums and land on the mirror, water spray is also present during the use of a high speed drill from water that is used to cool and lubricate the drilling site. The accumulation of material and water droplets from the cooling spray, as well as saliva and the patient's breath fogging the mirror, all present the well-known problem of obscured mirror vision for the dentist.

While the steps of withdrawing the mirror from the patient's mouth, wiping it clean, re-inserting the mirror (which requires re-orientation by the dentist and re-acquiring his “target”) may be only a few seconds for experienced dentists, when those steps must be repeated 50 to 100 times in the course of a normal day, the cumulative effect is significant. That is, even if the withdrawal of the mirror, wiping it clean, re-inserting it and re-orienting the tool and mirror take only a few seconds, the cumulative effect, if accomplished dozens of times each procedure with multiple procedures being performed each day, will add a significant amount of time to accomplish activities that are not that productive.

In addition, the repetitive wiping of the mirror is a source of aggravation and additional stress to the dentist. Further, the delay can be much greater than a few seconds, depending on the level of experience or skill of the dentist or of the difficulty or working in a specific location in the patient's mouth. For example, if the procedure is toward the rear on the inside of the teeth, the dentist will rely heavily on the mirror. If the dentist must re-orient the mirror every time the mirror is removed, wiped, and re-inserted, the procedure becomes significantly longer. The problem is so significant that many dentists, particularly those experienced and skilled enough to be able to “feel” the procedure even when their visibility is impaired, will resist the mirror withdrawal, wiping, re-insertion and re-orientation process, depending on the kind of procedure and the status of the procedure when the vision is obscured. If, for instance, the visibility in the mirror becomes obstructed near the end of a particular step in the procedure, the dentist will often complete the step with impaired vision rather than take the time to remove the mirror to wipe it clean. This may lead to less than optimal results for the patient.

Given this well-known problem, it is common for a dentist to employ a dental assistant during certain procedures and the assistant will use an air-water syringe device to clean the mirror with water and or air, thereby allowing the dentist to view the work surface without serious interruption. The air-water syringe is a standard piece of equipment for most dental offices and is capable of supplying air and/or water to the end of a flexible tube by pressing an air supply control mechanism (e.g., button) or a water supply control mechanism (e.g., button) and the air and water can be used to rinse and clean surfaces, including the mirrored surface of the dental instrument.

While this solution is very practical, it tends to increase the cost of providing dental services because the dental assistant must be present during the procedure, thereby adding additional costs to the procedure. Further, the addition of another person in the procedure room means that the procedure room must be large enough to accommodate the additional person. All of this may me impractical for one or more reasons. Accordingly, without improvements in the tools used in certain dental procedures, such as dental instruments, the time and energy spent by dental professionals performing those procedures will continue to be sub-optimal.

### BRIEF SUMMARY OF THE INVENTION

The present invention comprises two separate but cooperating elements. First, a dental instrument with a hollow body is attached to a flexible tube and the flexible tube is attached to the outlet or nozzle of a standard air-water syringe at the dentist's workstation. Additionally, a small locking mechanism is affixed to the standard control handle of an air-water syringe. The locking mechanism has a collar portion and a raised portion. The collar portion is designed to engage the neck of the water supply button or similar control, thereby preventing the water supply button from being depressed. The locking mechanism may be placed in a first position, with the water supply button essentially disengaged and with the air supply button or control being unaffected.

The locking mechanism may then be rotated from the first position to a second position and the raised portion of the locking mechanism will be positioned over the air supply button, thereby “locking” the air supply button in the “on” position. With the air supply button locked in the “on” position, the air supply from the air-water syringe is delivered through the flexible tubing, to an inlet or opening of the hollow body of the dental instrument, and then through the hollow body where the air supply is directed across the face of the mirrored surface of the dental instrument.

In this fashion, a constant stream of air can be used to keep the dental instrument clean from foreign objects and other material that might obscure the dental professionals view of the work surface. When the need for the air supply to the dental instrument is over, the locking mechanism may be returned to the first position, thereby allowing the air supply button to be disengaged and the air supply from the air-water syringe will be discontinued until needed again. In another preferred embodiment of the present invention, the locking mechanism may be configured to allow for delivery of air or water to the mirrored surface of the hollow-bodied mirror.

### BRIEF DESCRIPTION OF THE SEVERAL VIEWS OF THE DRAWINGS

The preferred embodiments of the present invention will hereinafter be described in conjunction with the appended drawings, wherein like designations denote like elements, and

FIG. 1 is a perspective view of a hollow-body dental instrument and locking mechanism connected to an air-water syringe for use in accordance with a preferred exemplary embodiment of the present invention;

FIG. 2 is a perspective view of a locking mechanism in accordance with a preferred exemplary embodiment of the present invention;

FIG. 3A and FIG. 3B are side views of a locking mechanism selectively engaging the air supply and water supply buttons on an air-water syringe in accordance with a preferred exemplary embodiment of the present invention; and

FIG. 4 is a flow chart of a method for using a hollow-body dental instrument and locking mechanism connected to an air-water syringe in accordance with a preferred exemplary embodiment of the present invention.

### DETAILED DESCRIPTION OF THE INVENTION

A dental instrument with a hollow body is attached to a flexible tube and the flexible tube is attached to the supply outlet of a standard air-water syringe at the dentist's workstation. The locking mechanism may be placed in a first position, with the water supply button of the air-water syringe being essentially disengaged and with the air supply button being unaffected. The locking mechanism may then be rotated or moved from the first position to a second position and the raised portion of the locking mechanism being positioned over the air supply button, thereby “locking” the air supply button in the “on” position.

With the air supply button locked in the “on” position, the air supply from the air-water syringe is delivered through the flexible tubing, through the hollow body of the dental instrument, and then directed across the face of the mirrored surface of the dental instrument. The hollow body as a channel or conduit for the air supply to be delivered to the mirrored surface. The hollow body has an outlet that directs the air supply over the face of the mirrored surface, thereby cleaning any liquid or debris from the face of the mirrored surface.

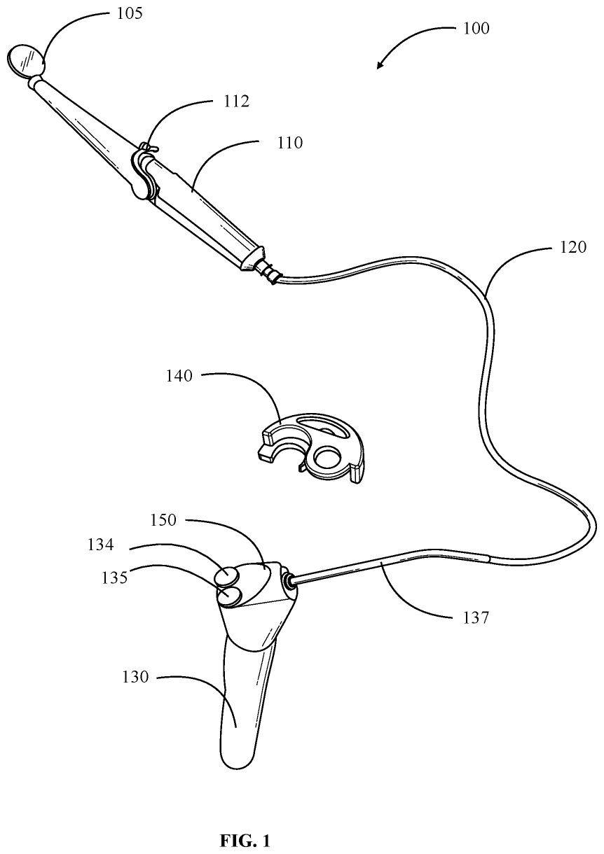

Referring now to FIG. 1, a perspective view of a system 100 for delivering air across the reflective surface of a hollow-body dental instrument in accordance with a preferred exemplary embodiment of the present invention is depicted. As shown in FIG. 1, a hollow-body dental instrument 110 is connected to a length of tubing 120. Tubing 120 is, in turn, connected to the outlet 137 of air-water syringe 130. Air-water syringe 130 has at least two buttons 134 and 135 that are used to selectively deliver air or water to outlet 137. Additionally, locking mechanism 140 is available to be attached to head portion 150 of air-water syringe 130 so as to control the flow of air or water to hollow-body dental instrument 110. In at least one preferred embodiment of the present invention, locking mechanism 140 may be removably attached to either of buttons 134 and 135.

When the dental professional wants to deliver air to the mirrored surface 105 of hollow-body dental instrument 110, locking mechanism 140 is attached to the head portion 150 of air-water syringe 130 so as to preliminarily engage at least one of the two buttons 134 and 135. In the most preferred embodiments of the present invention locking mechanism 140 will lock the air delivery button in the “on” position and the water delivery button in the “off” position. Additional information about the operation of locking mechanism 140 is presented below.

In addition, the most preferred embodiments of the present invention comprise a lever or switch 112 which can be use to temporarily shut off the flow of air through the hollow body of dental instrument 110, should the dental professional desires to use, position, or maneuver dental instrument 110 without directing an air stream across the reflective surface of dental instrument 110. Lever or switch 112 may be any type of lever or switch suitable to the task but is most preferably operable by a single finger or thumb. It may be a lever or switch that is selectively activated (e.g., by sliding a button or switch from a first position to a second position) or it may be some type of pressure or spring-activated lever or switch that is operated by pressing and hold the lever or switch in position.

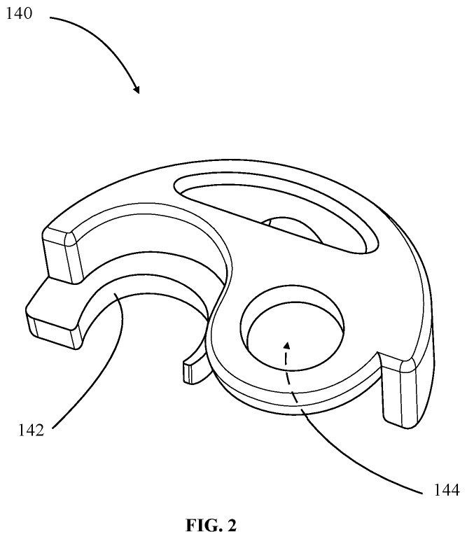

Referring now to FIG. 2, a perspective view of locking mechanism 140 in accordance with a preferred exemplary embodiment of the present invention is depicted. Locking mechanism 140 comprises a collar portion 142 and a raised portion 144 located on the underside of locking mechanism 140. Collar portion 142 snaps into place over the stem or neck of water supply button 340. The interior diameter of collar portion 142 is slightly larger than the exterior diameter of the stem or neck of water supply button 340. There is a small channel or cutout in collar portion 142 that allows collar portion 142 to be slipped into position and held in place by a friction fit. Collar portion 142 may be manufactured from any durable resilient material such as plastic, metal, etc.

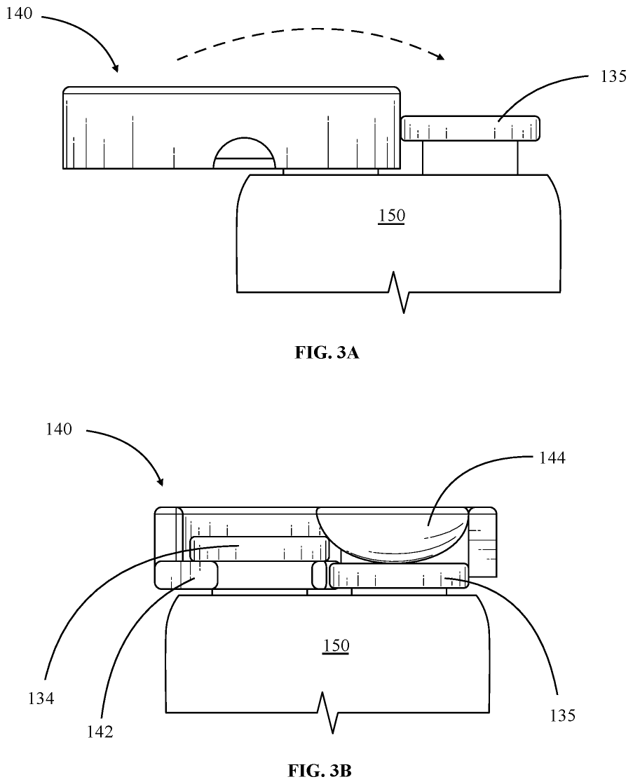

Referring now to FIG. 3A and FIG. 3B, side views of locking mechanism 140 selectively engaging the air supply and water supply controls or buttons (134 and 135, respectively) on the head portion 150 of an air-water syringe 130 in accordance with a preferred exemplary embodiment of the present invention are depicted. For the most preferred embodiments of the present invention, the body portion of dental instrument 110 is connected to outlet 137 of air-water syringe 130 by a length of tubing 120 as shown in FIG. 1. As shown in FIG. 3A, locking mechanism 140 is initially connected to a stem portion of water supply control 134 of an air-water syringe 130 and positioned away from air supply control 135.

The open collar portion 142 of locking mechanism 140 is “snapped” onto the stem portion of water supply control 340 and placed into a first position. Open collar portion 142 locking mechanism 140 is slightly smaller than the stem portion of water supply control 134. Since locking mechanism 140 is manufactured from a slightly resilient material such as plastic, there is a bit of “give” in the material, allowing open collar portion 142 to be forced onto the stem portion of water supply control 134 where it is rotatable around the stem portion of water supply control 134. In this configuration, locking mechanism 140 does not engage air supply control 135 and, according, no air is delivered to surface 105 of dental instrument 110 of FIG. 1.

As shown in FIG. 3B, locking mechanism 140 can be easily rotated from the first position to a second position where, in the second position, rounded portion 144 of locking mechanism 140 is forced over the top of air supply control 135, thereby depressing air supply control 135 and causing an air flow to be delivered to the hollow body of dental instrument 110 via tubing 120 and, subsequently, directed over mirrored surface 105 of dental instrument 110. The air flow will clean liquid and other debris from mirrored surface 105 of dental instrument 110, thereby allowing the dental professional to have a relatively unobstructed view into the patient's mouth.

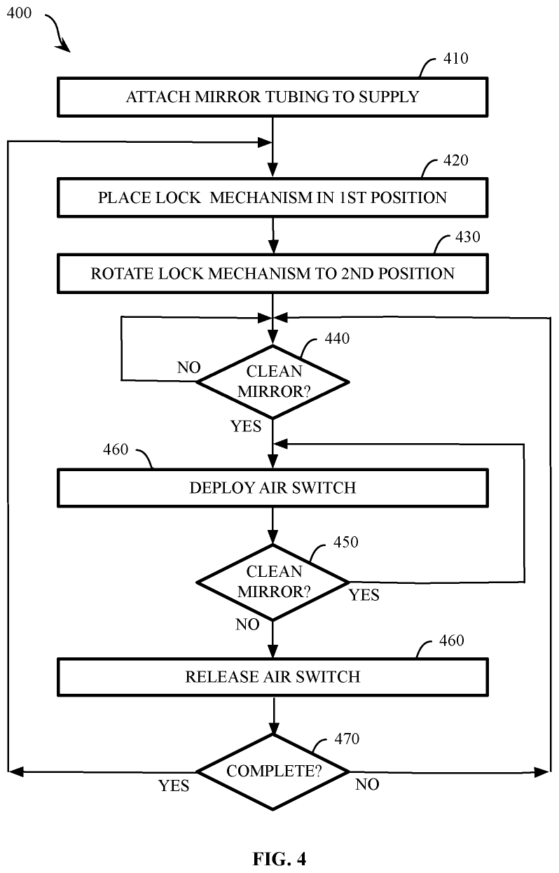

Referring now to FIG. 4, a flow chart of a method 400 for using a hollow-body dental instrument and locking mechanism connected to the water supply control of an air-water syringe in accordance with a preferred exemplary embodiment of the present invention is depicted. As shown in FIG. 4, the dental professional will attach the mirror to the output of the air-water syringe with a piece of tubing (step 410). With the tubing in place, the locking mechanism can be attached to the air-water syringe and placed into the first position (step 420). When the dental professional is ready to proceed with a specific procedure that will require the use of a mirror, the locking mechanism can be rotated into the second position where the water supply control is “locked” into the off position and the air supply control is “locked” into the on position (step 430). During the dental procedure, when the dental professional determines it's necessary to clean the reflective surface of the hollow body mirror (step 440=“Yes”) then the dental professional will deploy the air switch on the body of the mirror, thereby actuating the flow of air to the mirrored surface of the hollow body mirror as the air from the air-water syringe is delivered from the air supply of the air-water syringe to the mirrored surface of the mirror via the hollow body of the mirror. If the reflective surface of the mirror does not need to be cleaned (step 450=“No”) then the air switch on the body of the hollow-bodied mirror is not activated.

At anytime during the dental procedure when the dental professional determines that it is no longer necessary to clean the mirrored surface of the mirror (step 450=“No”) then the dental professional would release the air switch located on the body of the mirror (step 460). Then, once the dental procedure has been completed (step 470=“Yes”), then the locking mechanism can be returned to the first position (step 420). Otherwise, the dental professional will continue the dental procedure and clean the reflective surface of the mirror (step 440) as necessary.

Those skilled in the art will recognize that the various preferred embodiments of the present invention may be manufactured, maintained, repaired, and distributed in a manner similar to other dental tools and instruments known in the dental profession. For example, in at least one preferred embodiment of the present invention, the hollow-body dental instrument may be manufactured from a durable material such as stainless steel that is suitable for cleaning in an autoclave or, in an alternative preferred embodiment of the present invention, the hollow-body dental instrument may be manufactured from a very inexpensive material such as plastic so that it may be disposed of after each use so as to avoid the cost and time associated with using an autoclave.

Similarly, the exact parameters for the size and shape of the hollow-body dental instrument is flexible enough to encompass a wide variety of mirrors, depending on the preference of the dental professional and the specific dental procedure contemplated. In this fashion, there may be a wide variety of hollow-body dental instruments provided for various procedures and the dental professional can select the best mirror for the procedure.

The benefits of the present invention include time savings for all involved, the dentist, staff, and patient, because the time required to perform virtually every procedure will be reduced when there is no longer a need to continually remove the dental instrument, wipe it clean, reinsert it and get reoriented to the location for the procedure.

From the foregoing description, it should be appreciated that apparatus and method disclosed herein presents significant benefits that would be apparent to one skilled in the art. Furthermore, while multiple embodiments have been presented in the foregoing description, it should be appreciated that a vast number of variations in the embodiments exist. Lastly, it should be appreciated that these embodiments are preferred exemplary embodiments only and are not intended to limit the scope, applicability, or configuration of the invention in any way. Rather, the foregoing detailed description provides those skilled in the art with a convenient road map for implementing a preferred exemplary embodiment of the invention, it being understood that various changes may be made in the function and arrangement of elements described in the exemplary preferred embodiment without departing from the spirit and scope of the invention as set forth in the appended claims.

1. An apparatus comprising:
a locking mechanism, the locking mechanism comprising:
an open collared portion, the open collared portion being configured to engage a stem portion of a water supply control for an air-water syringe; and
a rounded portion, the rounded portion being configured to engage and depress an air supply control for the air-water syringe; and
a hollow-bodied dental instrument, the hollow-bodied dental instrument comprising:
a hollow body;
a mirrored surface;
an inlet configured to receive an air supply from a length of tubing; and
an outlet configured to deliver the air supply from the length of tubing to the mirrored surface via the hollow-bodied dental instrument.

2. The apparatus of claim 1 wherein the hollow-bodied mirror further comprises a switch, the switch being configured to selectively enable or disable the air supply being delivered to the mirrored surface.

3. The apparatus of claim 1 wherein the length of tubing is attached to an outlet of an air-water syringe.

4. The apparatus of claim 1 wherein the hollow-bodied dental instrument is manufactured from stainless steel.

5. The apparatus of claim 1 wherein the locking mechanism is manufactured from plastic.

6. The apparatus of claim one wherein the locking mechanism is removably attached to an air-water syringe by the open collared portion, the open collared portion engaging the stem portion of a water supply control mechanism on the air-water syringe.

7. The apparatus of claim one wherein the locking mechanism is removably attached to an air-water syringe in a first position and then moved to a second position wherein at least a portion of the locking mechanism engages and depresses the air supply control mechanism.

8. The apparatus of claim 1 further comprising an air-water syringe coupled to the dental instrument by the length of tubing.

9. The apparatus of claim 8 wherein the air-water syringe comprises a water supply button and an air supply button wherein the locking mechanism is positioned so as to prevent the water supply button from being depresses and simultaneously locking the air supply button into a position where the air supply is continuously delivered to the hollow-bodied dental instrument.

10. An apparatus for delivering an air supply to a mirrored surface, the apparatus comprising:
an air-water syringe, the air-water syringe comprising:
an air supply button configured to deliver air from the air-water syringe;
a water supply button configured to deliver water from the air-water syringe, the supply button comprising a stem portion; and
a nozzle configured to selectively deliver air or water from the air-water syringe;
a locking mechanism, the locking mechanism comprising:
an open collared portion, the open collared portion engaging the stem portion of the water supply button for an air-water syringe to prevent the water supply button from being depressed; and
a rounded portion, the rounded portion engaging and depressing the air supply button for the air-water syringe, thereby delivering air from the air-water syringe;
a hollow-bodied dental instrument, the hollow-bodied dental instrument comprising:
a hollow body;
a mirrored surface;
an inlet communicatively coupled to hollow body and configured to receive the from the air-water syringe; and
an outlet communicatively coupled to hollow body and configured to deliver the air to the mirrored surface via the hollow body; and
a length of tubing, the length of tubing connecting the nozzle of the air-water syringe to the inlet of the hollow-bodied dental instrument.

11. A method for supplying air, the method comprising the steps of:
attaching a first end of a length of tubing to an inlet for a hollow-bodied dental instrument, the hollow-bodied dental instrument comprising a mirrored surface;
attaching a second end of the length of tubing to a nozzle for an air-water syringe, the air-water syringe comprising:
a water supply control; and
an air supply control;
attaching a locking mechanism to a stem portion of the water supply control, the locking mechanism comprising:
an open collared portion, the open collared portion being configured to engage the stem portion of the water supply control; and
a rounded portion;
moving the locking mechanism from a first position into a second position wherein the rounded portion of the locking mechanism engages and actuates the air supply control; and
supplying air to a mirrored surface of the hollow-bodied dental instrument via the length of tubing.

12. The method of claim 11 further comprising the step of selectively activating a switch on the hollow-bodied dental instrument to temporarily disable the flow of air to the mirrored surface of the hollow-bodied dental instrument.

13. The method of claim 11 further comprising the step of moving the locking mechanism from the second position to the first position, thereby disengaging the rounded portion of the locking mechanism from the air supply control.
